(19) United States
(12) Patent Application Publication
(10) Pub. No.: US 2005/0244480 A1
(43) Pub. Date: Nov. 3, 2005

(54) Pre-wipes for improving anal cleansing

(75) Inventors: David W. Koenig, Menasha, WI (US); Duane G. Krzysik, Appleton, WI (US); David G. Biggs, Neenah, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/836,419

(22) Filed: Apr. 30, 2004

(51) Int. Cl. A61K 9/70

(52) U.S. Cl. 424/443

(57) ABSTRACT

Novel pre-wipes and methods are disclosed for assisting in the cleaning of skin in the anal area. The pre-wipes comprise an anti-adherent formulation and are wiped across the anal region of a user prior to defecation to introduce a film of the anti-adherent formulation onto the anal region. This film reduces the amount of fecal material that is retained in the anal region after defecation and reduces the amount of cleanup required. This reduced amount of cleanup results in cleaner, healthier skin.

### BACKGROUND OF INVENTION

The present invention generally relates to bath tissue products for improving anal cleansing. More particularly, the present invention relates to a pre-wipe bath tissue product comprising an anti-adherent formulation. The pre-wipe product is wiped across the anal region prior to defecation to introduce an anti-adherent film onto the region to minimize cleanup after defecation.

Over the course of many years a number of commercially available products have been developed to assist individuals in cleaning themselves thoroughly after a bowel movement. Conventional bath tissues have been used for many years and, recently, flushable wet bath tissues have been introduced. These products may be used alone, or in combination, to effectuate cleansing of the anal area after a bowel movement.

Because properly cleaning skin in the perianal, uro-genital, and vaginal regions may be difficult due to the topography of the skin in that region and the presence of hair follicles, a common problem encountered by many individuals during bowel movement cleanup is the occasional sticking of fecal material to the skin in the perianal and related regions. This sticking can result in a number of undesirable situations including, for example, transfer of the fecal material to undergarments and unwanted odors. Additionally, because fecal material generally contains bacteria and active enzymes, the presence of this material in the anal region after bowel movement cleanup can also result in skin irritation, redness, and even inflammation and infection for sensitive individuals. This problem may be particularly acute for individuals that defecate very frequently due to disease or illness.

Based on the foregoing, it is clear that maintaining clean and healthy skin in the perianal and surrounding areas is difficult, yet important. As such, products that can improve cleaning of the skin in these regions after a bowel movement are highly desirable. It would also be desirable for the products to be flushable and of low cost.

### SUMMARY OF THE INVENTION

The present invention is directed to novel pre-wipes which contain an anti-adherent formulation on a base substrate. The pre-wipes are wiped across the anal region prior to a bowel movement to impart an anti-adherent formulation onto the anal region such that, upon defecation, a reduced amount of fecal matter is retained on the skin in the anal and surrounding areas. This results in a reduced amount of required cleanup after a bowel movement, which can result in healthier, cleaner skin. The anti-adherent formulation is comfortable to use, and may comprise a pleasant fragrance and/or color.

The present invention is also directed to methods for cleaning skin in the anal area. These methods comprise wiping the anal area with a pre-wipe comprised of a base substrate and an anti-adherent formulation prior to having a bowel movement. This wiping introduces the anti-adherent formulation onto the skin in the anal region and results in a reduced amount of fecal matter being retained on the skin in this area. Because a reduced amount of fecal matter is retained on the skin in the anal area, post-bowel movement cleanup is reduced.

As such, the present invention is directed to a pre-wipe for assisting in anal cleansing. The pre-wipe comprises a base substrate and a hydrophobic anti-adherent formulation. The anti-adherent formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

The present invention is further directed to a pre-wipe for assisting in anal cleansing. The pre-wipe comprises a base substrate and a hydrophilic anti-adherent formulation. The anti-adherent formulation comprises from about 30% (by weight) to about 79.98% (by weight) glycol, from about 10% (by weight) to about 58% (by weight) polyethylene glycol having a melting point greater than 35° C., from about 10% (by weight) to about 58% (by weight) fatty acid or fatty alcohol, from about 0.01% (by weight) to about 10% (by weight) dimethicone or dimethiconol, and from about 0.01% (by weight) to about 1% (by weight) anti-adherent compound.

The present invention is further directed to a pre-wipe for assisting in anal cleansing. The pre-wipe comprises a base substrate and an anti-adherent formulation. The anti-adherent formulation comprises from about 99% (by weight) to about 99.99% (by weight) fatty acid ester having a melting point greater than 35° C. and from about 0.01% (by weight) to about 1% (by weight) anti-adherent compound.

The present invention is further directed to a pre-wipe for assisting in anal cleansing. The pre-wipe comprises a base substrate and an anti-adherent formulation. The anti-adherent formulation comprises from about 99% (by weight) to about 99.99% (by weight) Dow Corning 7-3076 polyamide blend and from about 0.01% (by weight) to about 1% (by weight) anti-adherent compound.

The present invention is further directed to a process for reducing the amount of cleansing of the anal area required after a bowel movement. The process comprises wiping the anal area with a pre-wipe prior to having a bowel movement. The pre-wipe comprises a substrate material and an anti-adherent formulation.

Other features of the present invention will be in part apparent and in part pointed out hereinafter.

### DETAILED DESCRIPTION OF THE PREFERRED EMBODIMENT

The present invention is generally directed to products and methods for assisting in the cleaning of skin in the anal area. Specifically, the present invention relates to novel pre-wipes comprising a base substrate and an anti-adherent formulation. When utilized prior to a bowel movement, the pre-wipes transfer an anti-adherent formulation onto the skin in the anal area of the user. This formulation may minimize the amount of fecal material that remains on the skin after a bowel movement and may make the removal of fecal material easier, which results in a reduced amount of cleanup and healthier, cleaner skin.

As noted above, the pre-wipes described herein comprise a base substrate and an anti-adherent formulation. Base substrates suitable for use in the pre-wipes of the present invention can be made from various materials and fibers, and are desirably soft to the touch. Optionally, the pre-wipes described herein may be flushable after use. The pre-wipe may be dry or wet to the touch, and may feel like conventional bath tissue, or like a wet wipe. The base substrate can be made from pulp fibers, other natural fibers, cellulose fibers, synthetic fibers such as polypropylene or polylactic acid, and the like. The base substrate may be woven or nonwoven and may be sized for easy single handed use. Although size is not critical, a suitable size may be, for example, 6 inches by about 4 inches.

One desirable base substrate is a tissue product substrate. The present invention is useful with tissue products and tissue paper in general, including but not limited to conventionally felt-pressed tissue paper, high bulk pattern densified tissue paper, and high bulk, uncompacted tissue paper. The tissue paper can be of a homogenous or multi-layered construction, and tissue paper products made therefrom can be of a single-ply or multi-ply construction. The tissue paper desirably has a basis weight of between about 10 g/m² and about 65 g/m², and a density of about 0.6 g/cc or less. More desirably, the basis weight will be about 40 g/m² or less and the density will be about 0.3 g/cc or less. Most desirably, the density will be between about 0.04 g/cc and about 0.2 g/cc. Unless otherwise specified, all amounts and weights relative to the paper are on a dry basis. Stretch in the machine direction can be in the range of from about 5% to about 20%. Stretch in the cross-machine direction can be in the range of from about 3% to about 20%. Tensile strengths in the machine direction can be in the range of from about 100 to about 5,000 grams per inch of width. Tensile strengths in the cross-machine direction are in the range of from about 50 grams to about 2,500 grams per inch of width. Absorbency is typically from about 5 grams of water per gram of fiber to about 9 grams of water per gram of fiber.

Conventionally pressed tissue paper and methods for making such paper are well known in the art. Such paper is typically made by depositing a papermaking furnish on a foraminous forming wire, often referred to in the art as a Fourdrinier wire. Once the furnish is deposited on the forming wire, it is referred to as a web. The web is dewatered by pressing the web and drying at an elevated temperature. The particular techniques and typical equipment for making webs according to the process just described are well known to those skilled in the art. In a typical process, a low consistency pulp furnish is provided from a pressurized headbox, which has an opening for delivering a thin deposit of pulp furnish onto the Fourdrinier wire to form a wet web. The web is then typically dewatered to a fiber consistency of between about 7% and about 25% (total web weight basis) by vacuum dewatering and further dried by pressing operations wherein the web is subjected to pressure developed by opposing mechanical members, for example, cylindrical rolls. The dewatered web is then further pressed and dried by a steam drum apparatus known in the art as a Yankee dryer. Pressure can be developed at the Yankee dryer by mechanical means such as an opposing cylindrical drum pressing against the web. Multiple Yankee dryer drums can be employed, whereby additional pressing is optionally incurred between the drums. The formed sheets are considered to be compacted since the entire web is subjected to substantial mechanical compressional forces while the fibers are moist and are then dried while in a compressed state.

High bulk pattern densified tissue paper is characterized by having a relatively high bulk field of relatively low fiber density and an array of densified zones of relatively high fiber density. The high bulk field is alternatively characterized as a field of pillow regions. The densified zones are alternatively referred to as knuckle regions. The densified zones can be discretely spaced within the high bulk field or can be interconnected, either fully or partially, within the high bulk field. The patterns can be formed in a non-ornamental configuration or can be formed so as to provide an ornamental design(s) in the tissue paper. Preferred processes for making pattern densified tissue webs are disclosed in U.S. Pat. No. 3,301,746 (Sanford et al.), issued Jan. 31, 1967; U.S. Pat. No. 3,974,025 (Ayers), issued Aug. 10, 1976; and U.S. Pat. No. 4,191,609 (Trokhan), issued Mar. 4, 1980; and U.S. Pat. No. 4,637,859 (Trokhan), issued Jan. 20, 1987; all of which are incorporated by reference.

In general, pattern densified webs are preferably prepared by depositing a papermaking furnish on a foraminous forming wire such as a Fourdrinier wire to form a wet web and then juxtaposing the web against an array of supports. The web is pressed against the array of supports, thereby resulting in densified zones in the web at the locations geographically corresponding to the points of contact between the array of supports and the wet web. The remainder of the web not compressed during this operation is referred to as the high bulk field. This high bulk field can be further de-densified by application of fluid pressure, such as with a vacuum type device or a blow-through dryer, or by mechanically pressing the web against the array of supports. The web is dewatered, and optionally predried, in such a manner so as to substantially avoid compression of the high bulk field. This is preferably accomplished by fluid pressure, such as with a vacuum type device or blow-through dryer, or alternately by mechanically pressing the web against an array of supports wherein the high bulk field is not compressed. The operations of dewatering, optional predrying and formation of the densified zones can be integrated or partially integrated to reduce the total number of processing steps performed. Subsequent to formation of the densified zones, dewatering, and optional predrying, the web is dried to completion, preferably still avoiding mechanical pressing. Preferably, from about 8% to about 55% of the tissue paper surface comprises densified knuckles having a relative density of at least 125% of the density of the high bulk field.

Desirably, the furnish is first formed into a wet web on a foraminous forming carrier, such as a Fourdrinier wire. The web is dewatered and transferred to an imprinting fabric. The furnish can alternately be initially deposited on a foraminous supporting carrier that also operates as an imprinting fabric. Once formed, the wet web is dewatered and, preferably, thermally pre-dried to a selected fiber consistency from about 40% to about 80%. Dewatering is preferably performed with suction boxes or other vacuum devices or with blow-through dryers. The knuckle imprint of the imprinting fabric is impressed in the web as discussed above, prior to drying the web to completion. One method for accomplishing this is through application of mechanical pressure. This can be done, for example, by pressing a nip roll that supports the imprinting fabric against the face of a drying drum, such as a Yankee dryer, wherein the web is disposed between the nip roll and drying drum. Also, preferably, the web is molded against the imprinting fabric prior to completion of drying by application of fluid pressure with a vacuum device such as a suction box, or with a blow-through dryer. Fluid pressure can be applied to induce impression of densified zones during initial dewatering, in a separate, subsequent process stage, or a combination thereof.

Uncompacted, nonpattern-densified tissue paper structures are described in U.S. Pat. No. 3,812,000 (Salvucci et al.), issued May 21, 1974 and U.S. Pat. No. 4,208,459 (Becker et al.), issued Jun. 17, 1980, both of which are incorporated by reference. In general, uncompacted, nonpattern-densified tissue paper structures are prepared by depositing a papermaking furnish on a foraminous forming wire such as a Fourdrinier wire to form a wet web, draining the web and removing additional water without mechanical compression until the web has a fiber consistency of at least about 80%, and creping the web. Water is removed from the web by vacuum dewatering and thermal drying. The resulting structure is a soft but weak, high bulk sheet of relatively uncompacted fibers. Bonding material is preferably applied to portions of the web prior to creping.

Compacted non-pattern-densified tissue structures are commonly known in the art as conventional tissue structures. In general, compacted, non-pattern-densified tissue paper structures are prepared by depositing a papermaking furnish on a foraminous wire such as a Fourdrinier wire to form a wet web, draining the web and removing additional water with the aid of a uniform mechanical compaction (pressing) until the web has a consistency of 25-50%, transferring the web to a thermal dryer such as a Yankee dryer and creping the web. Overall, water is removed from the web by vacuum, mechanical pressing and thermal means. The resulting structure is strong and generally of singular density, but very low in bulk, absorbency and softness.

The papermaking fibers utilized in preparing tissue paper for the products of the present invention will normally include fibers derived from wood pulp. Other cellulosic fibrous pulp fibers, such as cotton linters, bagasse, etc., can be utilized and are intended to be within the scope of this invention. Synthetic fibers, such as rayon, polyethylene and polypropylene fibers, can also be utilized in combination with natural cellulosic fibers. One exemplary polyethylene fiber that can be utilized is Pulpex.RTM., available from Hercules, Inc. (Wilmington, Del.).

Applicable wood pulps include chemical pulps, such as Kraft, sulfite, and sulfate pulps, as well as mechanical pulps including, for example, groundwood, thermo-mechanical pulp and chemically modified thermo-mechanical pulp. Chemical pulps, however, are typically desirable since they impart a superior tactile sense of softness to tissue sheets made therefrom. Pulps derived from both deciduous trees and coniferous trees can be utilized. Also useful in the present invention are fibers derived from recycled paper, which can contain any or all of the above categories as well as other non-fibrous materials such as fillers and adhesives used to facilitate the original papermaking.

In addition to papermaking fibers, the papermaking furnish used to make tissue paper structures can have other components or materials added thereto as can be or later become known in the art. The types of additives desirable will be dependent upon the particular end use of the tissue sheet contemplated. For example, in products such as bath tissue, paper towels, facial tissues and other similar products, adequate wet strength is a desirable attribute. Thus, it is often desirable to add to the papermaking furnish chemical substances known in the art as “wet strength” additives.

In addition to wet strength additives, it can also be desirable to include in the papermaking fibers certain dry strength and lint control additives known in the art. In this regard, starch binders have been found to be particularly suitable. In addition to reducing tinting of the finished tissue paper product, low levels of starch binders also impart a modest improvement in the dry tensile strength without imparting stiffness that could result from the addition of high levels of starch. Typically, the starch binder is included in an amount such that it is retained at a level of from about 0.01 to about 2%, preferably from about 0.1 to about 1%, by weight of the dry tissue paper.

As noted above, in some embodiments, the pre-wipes described herein are flushable pre-wipes suitable for disposal in conventional toilets. A description of properties suitable for dispersible (flushable) wipe products is set forth in U.S. Published patent application Ser. No. 20020155281 A1, which is incorporated herein by reference. Typically, it is desirable for the pre-wipe substrate to rapidly disperse in an aqueous environment, so as to enhance the flushability of the pre-wipe after use. Substrates suitable for this embodiment tend to have greatly diminished wet strengths (approaching zero), as compared to their dry strengths, after they are saturated for as little as several seconds. Having adequate in-use dry strength is desirable so as to avoid poke-through or tearing of the pre-wipe in use, and the soiling of hands.

Tissue paper products suitable for disposal in mechanical toilets are typically designed to easily disperse with mild agitation in a toilet bowl. These products tend to easily evacuate the toilet bowl and aid in the subsequent handling in domestic and residential sewer and septic systems. Non-woven substrates can also be fabricated to be substantially dispersible when manufactured with water dispersible binders. Water dispersible binders used in the production of non-wovens are particularly suitable as the anti-adherent formulations described herein are substantially non-aqueous, and thus will not weaken the product prior to disposal.

The formulations described herein for use in combination with the tissue product are either solid or semi-solid at room temperature. As used herein, the term semi-solid means that the formulation has a rheology typical of pseudoplastic or plastic fluids. When applied to the tissue product, the formulations described herein impart a soft, lubricious, lotion-like feel to the touch. The formulation is transferred to the skin of the user upon use to improve the skin health of the user.

The base substrates described above comprise an anti-adherent formulation thereon. The anti-adherent formulation may be hydrophobic or hydrophilic. This formulation is transferred to the anal area of a user when the pre-wipe is wiped or rubbed across this area prior to a bowel movement. The formulation forms film on the skin. This film may result in a reduced amount of fecal material remaining on the skin after defecation. Additionally, the film may result in easier removal of any fecal material that does remain on the skin. This can result in healthier, cleaner skin.

Additionally, the pre-wipes described herein can be utilized in the anal area by a user after the cleanup from a bowel movement is complete; that is, the pre-wipes can be used after a bowel movement cleanup such that a user would not have to use a pre-wipe immediately before the next bowel movement. This may be a convenient way of reducing cleanup for some users who may not have complete control of their bowels, or those who defecate very frequently.

In one embodiment, the anti-adherent formulation comprises a hydrophobic anti-adherent formulation. In this embodiment, the formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

An emollient is an active ingredient in a formulation that typically softens, soothes, supples, coats, lubricates and/or moisturizes the skin. Generally, emollients accomplish several of these objectives simultaneously. Typically, emollients suitable for use in the anti-adherent formulations described herein are fluids at room temperature such that they impart a soft, lubricious lotion-like feel upon use. Suitable emollients for use in the formulations of the present invention are typically substantially water free. Although the emollient component may contain trace amounts of water as a contaminant without substantially harming the formulation, it is preferred that the amount of water be less than about 5% by weight of the emollient component of the formulation to reduce the likelihood of microbial growth and product destruction.

Suitable emollients for inclusion in the formulations described herein include petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof. Petrolatum and mineral oil are preferred emollients.

The structuring agent utilized in the anti-adherent formulations described herein helps to immobilize the emollient and other components on the surface of the base substrate where they are of greatest value. Because some emollients are fluids at room temperature, they may tend to flow or migrate away from the surface of the base substrate and into the interior of the base substrate where they are of limited value due to non-transferability, and may tend to decrease the strength of the base substrate due to debonding. The structuring agent reduces the ability of the emollient (and other components) to migrate and keeps the emollient primarily on the surface of the base substrate.

Suitable structuring agents include animal waxes, vegetable waxes, mineral waxes, synthetic waxes, polymers, bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

The rheology modifier utilized in the anti-adherent formulation increases the melt point viscosity of the formulation so that the formulation readily remains on the surface of the base substrate and does not substantially migrate into the interior of the base substrate, while substantially not affecting the transfer of the anti-adherent formulation to the skin. Additionally, the rheology modifier helps the anti-adherent formulation to maintain a high viscosity at elevated temperatures, such as those encountered during storage and transportation.

Suitable rheology modifiers include combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, styrene-ethylene/propylene copolymers, and silicas.

The anti-adherent compound included in the anti-adherent formulations described herein acts to prevent the adherence of fecal material to the skin in the anal region during and after defecation. The presence of the anti-adherent compound in the formulation results in a decreased amount of fecal material in the anal region after a bowel movement. Without being bound to a particular theory, it is believed that the anti-adherent compound attaches to the skin through electrical interaction with the skin and remains tightly bound thereto after deposit. When defecation occurs, bacteria and enzymes in the fecal material, which also typically attach to skin through electrical interactions, are not able to make the attachment to the skin as many of the binding sites are already occupied with anti-adherent compound. Because electrical interaction with the bacteria and enzymes and the skin is reduced, much less fecal matter remains attached to the skin after defecation.

Suitable anti-adherent compounds include alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

The hydrophobic anti-adherent formulation described above may have specific melt point and process temperature viscosities, as defined herein. These viscosities are important for at least two reasons. First, the higher the melt point or process temperature viscosity, the less likely the anti-adherent formulation is to penetrate into the inner surface of the base substrate. The less formulation that is able to penetrate into the interior of the base substrate, results in more formulation on the surface of the base substrate that can transfer to the user's anal skin. Secondly, the higher the viscosity of the formulation at or above the melting point of the formulation, the less likely the formulation will be to migrate at typical or adverse storage or temperature conditions.

The hydrophobic anti-adherent formulations described above have a melt point viscosity of from about 5000 cPs to about 1,000,000 cPs, desirably from about 50,000 cPs to about 800,000 cps, and more desirably from about 100,000 cPs to about 500,000 cps. As used herein, the term “melt point viscosity” means the viscosity of the formulation at the point in time when the formulation visually becomes a liquid. Formulations having melt point viscosities in these ranges significantly improve the ability of the formulation to remain on the surface of the base substrate and the formulation maintains a high viscosity at elevated temperatures, such as those encountered during storage and shipment.

Additionally, to improve application to the surface of the base substrate, the hydrophobic formulations described herein have a process temperature viscosity of from about 50 cPs to about 50,000 cps, desirably from about 75 cPs to about 10,000 cps, and more desirably from about 100 cPs to about 5,000 cps. The process temperature is typically from about 5° C. to about 10° C. above the melting point of the lotion formulation.

In an alternative embodiment of the present invention, the anti-adherent formulation may comprise a hydrophilic formulation. The hydrophilic formulation may comprise from about 30% (by weight) to about 79.98% (by weight) glycol, from about 10% (by weight) to about 58% (by weight) polyethylene glycol having a melting point greater than about 35° C., from about 10% (by weight) to about 58% (by weight) fatty acid or fatty alcohol, from about 0.01% (by weight) to about 10% (by weight) dimethicone or dimethiconol, and from about 0.01% (by weight) to about 1% (by weight) anti-adherent compound.

The glycol component of the hydrophilic anti-adherent formulation acts to ensure a high degree of compatibility between the components and ensures that a homogeneous formulation is produced. Suitable glycols include, for example, propylene glycol, butylene glycol, 1,3 butylene glycol, polyethylene glycols that are liquid at room temperature, dipropylene glycol, methylpropane glycol, silicone glycol, polypropylene glycol, hydrogenate starch hydrolysates, and combinations thereof. Polyethylene glycols that are liquid at room temperature include low molecular weight polyethylene glycols, such as those having a molecular weight of less than about 720 (e.g., PEG 600).

Polyethylene glycols having a melting point greater than about 35° C. are included in the anti-adherent formulation as structurants. Suitable polyethylene glycols in this category include polyethylene glycols having a molecular weight greater than about 720.

The fatty acid or fatty alcohol is included in the anti-adherent formulation as structurants and emollients. Suitable fatty acids or fatty alcohols include those having a carbon chain length of from about 14 to about 22 carbon atoms. Specific examples include myristyl alcohol, cetyl alcohol, stearyl alcohol, and behenyl alcohol.

The dimethicone or dimethiconol is included in the formulation as an emollient. Suitable examples include, for example, Dow Corning 200 and Dow Corning 1503.

In another embodiment of the present invention, the anti-adherent formulation may comprise from about 99% (by weight) to about 99.99% (by weight) fatty acid ester having a melting point greater than 35° C. and from about 0.01% (by weight) to about 1% (by weight) anti-adherent compound. The fatty acid ester is a structurant and an emollient and may include compounds such as, for example, myristal myristate, cetyl palmitate, cetyl benzoate, cetyl lactate, steryl behenate, and combinations thereof.

In another embodiment of the present invention, the anti-adherent formulation may comprise from about 99% (by weight) to about 99.99% (by weight) Dow Corning 7-3076 polyamide blend and from about 0.01% (by weight) to about 1% (by weight) anti-adherent compound. The Dow Corning 7-3076 is a proprietary blend of Nylon 6111 dimethicone copolymer and PPG 3 myristal ether.

In still another embodiment of the present invention, the anti-adherent formulation may comprise one or more of the following components: petrolatum, glycerin, mineral oil, and olive oil.

In one embodiment of the present invention, the anti-adherent formulation, or one or more components of the anti-adherent formulation such as the anti-adherent compound, may be encapsulated in a shell material prior to being introduced onto the base substrate. When the pre-wipe is wiped across the anal region prior to defecation, the capsules break open due to the shear of the wiping and release the formulation or component(s). Additionally, the pre-wipes may be dispensed from a dispensing unit that, upon dispensing, creates shear and causes the capsules to break and release the formulation or component(s). Suitable microencapsulation shell materials include cellulose-based polymeric materials (e.g., ethyl cellulose), carbohydrate-based materials (e.g., cationic starches and sugars) and materials derived therefrom (e.g., dextrins and cyclodextrins) as well as other materials compatible with human tissues.

The microencapsulation shell thickness may vary depending upon the anti-adherent formulation utilized, and is generally manufactured to allow the encapsulated formulation or component to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should also be constructed such that humidity from atmospheric conditions during storage, shipment, or wear will not cause a breakdown of the microencapsulation layer and result in a release formulation or component.

Microencapsulated formulations or components applied directly to the pre-wipes should be of a size such that the user cannot feel the encapsulated shell on the skin during use. Typically, the capsules have a diameter of no more than about 25 micrometers, and desirably no more than about 10 micrometers. At these sizes, there is no “gritty” or “scratchy” feeling on the skin when the pre-wipe is utilized prior to defecation.

The pre-wipes described herein contain an amount of anti-adherent formulation such that, upon wiping across the anal region, an effective amount of formulation is transferred to the skin surface. Specifically, the pre-wipe may suitably contain from about 1% (by weight of the base substrate) to about 25% (by weight of the base substrate), desirably from about 1% (by weight of the base substrate) to about 10% (by weight of the base substrate). Based on the disclosure herein, one skilled in the art will recognize that various amounts of anti-adherent formulation may be suitable for different end products.

The anti-adherent formulations described herein can be introduced onto a suitable base substrate utilizing various techniques known in the art. For example, the anti-adherent formulation may include a suspending or thickening agent to suspend the formulation such that it can be gravure or flexographically coated, sprayed, ink-jet printed, or slot coated onto the base substrate in the desired amount. Suitable thickening agents may include, for example, clays, cellulose derivatives such as carboxymethyl cellulose and carboxypropyl cellulose, natural gums such as guar gum and xanthan gum, and acrylate polymers.

As will be recognized by one skilled in the art based on the disclosure herein, the pre-wipe products described herein can be manufactured and sold to consumers in various product forms. For example, the pre-wipes could be manufactured and sold in roll form, as individual sheets, or in stacks of individual sheets. In any of these forms, the pre-wipe product can be in wet form similar to a wet wipe, or could be dry to the touch such that a consumer would wet the product prior to use.

In addition to the components of the various anti-adherent formulations described herein, each formulation may additionally comprise one or more optional components to impart additional benefits to the anti-adherent formulations. Suitable optional components include, for example, skin protectants, powders, anti-biotics, anti-microbials, anti-inflammatories, fragrances, colorants, vitamin E, aloe extract, and preservatives.

In view of the above, it will be seen that the several objects of the invention are achieved and other advantageous results obtained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

1. A pre-wipe for assisting in anal cleansing comprising a base substrate and a hydrophobic anti-adherent formulation, the anti-adherent formulation comprising from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

2. The pre-wipe as set forth in claim 1 wherein the emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

3. The pre-wipe as set forth in claim 1 wherein the structuring agent is selected from the group consisting of animal waxes, vegetable waxes, mineral waxes, synthetic waxes, polymers, bayberry wax, beeswax, stearyl dimethicone, stearyl trimethicone, C_{2O}-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

4. The pre-wipe as set forth in claim 1 wherein the rheology modifier is selected from the group consisting of combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, and styrene-ethylene/propylene copolymers.

5. The pre-wipe as set forth in claim 1 wherein the rheology modifier is a silica.

6. The pre-wipe as set forth in claim 1 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

7. The pre-wipe as set forth in claim 1 wherein the anti-adherent formulation additionally comprises an optional component selected from the group consisting of skin protectants, powders, fragrances, colorants, and preservatives.

8. The pre-wipe as set forth in claim 1 wherein the base substrate comprises from about 1% (by weight of the base substrate) to about 25% (by weight of the base substrate) of the anti-adherent formulation.

9. The pre-wipe as set forth in claim 1 wherein the base substrate comprises from about 1% (by weight of the base substrate) to about 10% (by weight of the base substrate) of the anti-adherent formulation.

10. The pre-wipe as set forth in claim 1 wherein the anti-adherent formulation is encapsulated in a shell material.

11. The pre-wipe as set forth in claim 10 wherein the shell material comprises a material selected from the group consisting of cellulose-based polymeric materials, carbohydrate-based materials, and materials derived therefrom.

12. The pre-wipe as set forth in claim 1 wherein the base substrate comprises a material selected from the group consisting of cellulosic fibers and synthetic fibers.

13. The pre-wipe as set forth in claim 1 wherein the base substrate is a woven material.

14. The pre-wipe as set forth in claim 1 wherein the base substrate is a non-woven material.

15. The pre-wipe as set forth in claim 1 wherein the base substrate is a single ply substrate.

16. The pre-wipe as set forth in claim 1 wherein the base substrate is a multiple ply substrate.

17. The pre-wipe as set forth in claim 1 wherein both sides of the base substrate comprise the anti-adherent formulation.

18. The pre-wipe as set forth in claim 1 wherein one side of the base substrate comprises the anti-adherent formulation.

19. A pre-wipe for assisting in anal cleansing comprising a base substrate and a hydrophilic anti-adherent formulation, the anti-adherent formulation comprising from about 30% (by weight) to about 79.98% (by weight) of a glycol, from about 10% (by weight) to about 58% (by weight) of a polyethylene glycol having a melting point greater than 35° C., from about 10% (by weight) to about 58% (by weight) of a fatty acid or fatty alcohol, from about 0.01% (by weight) to about 10% (by weight) of a dimethicone or dimethiconol, and from about 0.01% (by weight) to about 1% (by weight) of an anti-adherent compound.

20. The pre-wipe as set forth in claim 19 wherein the glycol is selected from the group consisting of propylene glycol, butylene glycol, 1,3 butylene glycol, polyethylene glycols which are liquid at room temperature, dipropylene glycol, methylpropane glycol, silicon glycol, polypropylene glycol, hydrogenate starch hydrolysates, and combinations thereof.

21. The pre-wipe as set forth in claim 19 wherein the fatty acid has a carbon chain length of C₁₄-C₂₂.

22. The pre-wipe as set forth in claim 19 wherein the fatty alcohol has a carbon chain length of C₁₄-C₂₂.

23. The pre-wipe as set forth in claim 19 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

24. The pre-wipe as set forth in claim 19 wherein the anti-adherent formulation additionally comprises an optional component selected from the group consisting of skin protectants, powders, fragrances, colorants, and preservatives.

25. The pre-wipe as set forth in claim 19 wherein the base substrate comprises from about 1% (by weight of the base substrate) to about 25% (by weight of the base substrate) of the anti-adherent formulation.

26. The pre-wipe as set forth in claim 19 wherein the base substrate comprises from about 1% (by weight of the base substrate) to about 10% (by weight of the base substrate) of the anti-adherent formulation.

27. The pre-wipe as set forth in claim 19 wherein the anti-adherent formulation is encapsulated in a shell material.

28. The pre-wipe as set forth in claim 27 wherein the shell material comprises a material selected from the group consisting of cellulose-based polymeric materials, carbohydrate-based materials, and materials derived therefrom.

29. The pre-wipe as set forth in claim 19 wherein the base substrate comprises a material selected from the group consisting of cellulosic fibers and synthetic fibers.

30. The pre-wipe as set forth in claim 19 wherein the base substrate is a woven material.

31. The pre-wipe as set forth in claim 19 wherein the base substrate is a non-woven material.

32. The pre-wipe as set forth in claim 19 wherein the base substrate is a single ply substrate.

33. The pre-wipe as set forth in claim 19 wherein the base substrate is a multiple ply substrate.

34. The pre-wipe as set forth in claim 19 wherein both sides of the base substrate comprise the anti-adherent formulation.

35. The pre-wipe as set forth in claim 19 wherein one side of the base substrate comprises the anti-adherent formulation.

36. A pre-wipe for assisting in anal cleansing comprising a base substrate and an anti-adherent formulation, the anti-adherent formulation comprising from about 99% (by weight) to about 99.99% (by weight) of fatty acid ester having a melting point greater than 35° C. and from about 0.01% (by weight) to about 1% (by weight) of an anti-adherent compound.

37. The pre-wipe as set forth in claim 36 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

38. The pre-wipe as set forth in claim 36 wherein the anti-adherent formulation additionally comprises an optional component selected from the group consisting of skin protectants, powders, fragrances, colorants, and preservatives.

39. The pre-wipe as set forth in claim 36 wherein the base substrate comprises from about 1% (by weight of the base substrate) to about 25% (by weight of the base substrate) of the anti-adherent formulation.

40. The pre-wipe as set forth in claim 36 wherein the base substrate comprises from about 1% (by weight of the base substrate) to about 10% (by weight of the base substrate) of the anti-adherent formulation.

41. The pre-wipe as set forth in claim 36 wherein the anti-adherent formulation is encapsulated in a shell material.

42. The pre-wipe as set forth in claim 41 wherein the shell material comprises a material selected from the group consisting of cellulose-based polymeric materials, carbohydrate-based materials, and materials derived therefrom.

43. The pre-wipe as set forth in claim 36 wherein the base substrate comprises a material selected from the group consisting of cellulosic fibers and synthetic fibers.

44. The pre-wipe as set forth in claim 36 wherein the base substrate is a woven material.

45. The pre-wipe as set forth in claim 36 wherein the base substrate is a non-woven material.

46. The pre-wipe as set forth in claim 36 wherein the base substrate is a single ply substrate.

47. The pre-wipe as set forth in claim 36 wherein the base substrate is a multiple ply substrate.

48. The pre-wipe as set forth in claim 36 wherein both sides of the base substrate comprise the anti-adherent formulation.

49. The pre-wipe as set forth in claim 36 wherein one side of the base substrate comprises the anti-adherent formulation.

50. A pre-wipe for assisting in anal cleansing comprising a base substrate and an anti-adherent formulation, the anti-adherent formulation comprising from about 99% (by weight) to about 99.99% (by weight) of Dow Corning 7-3076 polyamide blend and from about 0.01% (by weight) to about 1% (by weight) of an anti-adherent compound.

51. The pre-wipe as set forth in claim 50 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

52. The pre-wipe as set forth in claim 50 wherein the anti-adherent formulation additionally comprises an optional component selected from the group consisting of skin protectants, powders, fragrances, colorants, and preservatives.

53. The pre-wipe as set forth in claim 50 wherein the base substrate comprises from about 1% (by weight of the base substrate) to about 25% (by weight of the base substrate) of the anti-adherent formulation.

54. The pre-wipe as set forth in claim 50 wherein the base substrate comprises from about 1% (by weight of the base substrate) to about 10% (by weight of the base substrate) of the anti-adherent formulation.

55. The pre-wipe as set forth in claim 50 wherein the anti-adherent formulation is encapsulated in a shell material.

56. The pre-wipe as set forth in claim 55 wherein the shell material comprises a material selected from the group consisting of cellulose-based polymeric materials, carbohydrate-based materials and materials derived therefrom.

57. The pre-wipe as set forth in claim 50 wherein the base substrate comprises a material selected from the group consisting of cellulosic fibers and synthetic fibers.

58. The pre-wipe as set forth in claim 50 wherein the base substrate is a woven material.

59. The pre-wipe as set forth in claim 50 wherein the base substrate is a non-woven material.

60. The pre-wipe as set forth in claim 50 wherein the base substrate is a single ply substrate.

61. The pre-wipe as set forth in claim 50 wherein the base substrate is a multiple ply substrate.

62. The pre-wipe as set forth in claim 50 wherein both sides of the base substrate comprise the anti-adherent formulation.

63. The pre-wipe as set forth in claim 50 wherein one side of the base substrate comprises the anti-adherent formulation.

64. A process for reducing the amount of cleansing of the anal area required after a bowel movement, the process comprising:
wiping the anal area with a pre-wipe prior to having a bowel movement, the pre-wipe comprising a substrate material and an anti-adherent formulation.

65. The process as set forth in claim 64 wherein the anti-adherent formulation comprises a lubricant selected from the group consisting of petrolatum, glycerin, mineral oil, olive oil, and combinations thereof.

66. The process as set forth in claim 64 wherein the anti-adherent formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

67. The process as set forth in claim 66 wherein the emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

68. The process as set forth in claim 66 wherein the structuring agent is selected from the group consisting of animal waxes, vegetable waxes, mineral waxes, synthetic waxes, polymers, bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

69. The process as set forth in claim 66 wherein the rheology modifier is selected from the group consisting of combinations of alpha-olefins and styrene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, and styrene-ethylene/propylene copolymers.

70. The process as set forth in claim 66 wherein the rheology modifier is a silica.

71. The process as set forth in claim 66 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

72. The process as set forth in claim 66 wherein the anti-adherent formulation additionally comprises an optional component selected from the group consisting of skin protectants, powders, fragrances, colorants, and preservatives.

73. The process as set forth in claim 66 wherein the base substrate comprises from about 1% (by weight of the base substrate) to about 25% (by weight of the base substrate) of the anti-adherent formulation.

74. The process as set forth in claim 66 wherein the base substrate comprises from about 1% (by weight of the base substrate) to about 10% (by weight of the base substrate) of the anti-adherent formulation.

75. The process as set forth in claim 66 wherein the anti-adherent formulation is encapsulated in a shell material.

76. The process as set forth in claim 75 wherein the shell material comprises a material selected from the group consisting of cellulose-based polymeric materials, carbohydrate-based materials, and materials derived therefrom.

77. The process as set forth in claim 66 wherein the base substrate comprises a material selected from the group consisting of cellulosic fibers and synthetic fibers.

78. The process as set forth in claim 66 wherein the base substrate is a woven material.

79. The process as set forth in claim 66 wherein the base substrate is a non-woven material.

80. The process as set forth in claim 66 wherein the base substrate is a single ply substrate.

81. The process as set forth in claim 66 wherein the base substrate is a multiple ply substrate.

82. The process as set forth in claim 66 wherein both sides of the base substrate comprise the anti-adherent formulation.

83. The process as set forth in claim 66 wherein one side of the base substrate comprises the anti-adherent formulation.

84. The process as set forth in claim 64 wherein the anti-adherent formulation comprises from about 30% (by weight) to about 79.98% (by weight) of a glycol, from about 10% (by weight) to about 58% (by weight) of a polyethylene glycol having a melting point greater than 35° C., from about 10% (by weight) to about 58% (by weight) of a fatty acid or fatty alcohol, from about 0.01% (by weight) to about 10% (by weight) of a dimethicone or dimethiconol, and from about 0.01% (by weight) to about 1% (by weight) of an anti-adherent compound.

85. The process as set forth in claim 84 wherein the glycol is selected from the group consisting of propylene glycol, butylene glycol, polyethylene glycol which are liquid at room temperature, dipropylene glycol, methylpropane glycol, silicon glycol, polypropylene glycol, and combinations thereof.

86. The process as set forth in claim 84 wherein the fatty acid has a carbon chain length of C₁₄-C₂₂.

87. The process as set forth in claim 84 wherein the fatty alcohol has a carbon chain length of C₁₄-C₂₂.

88. The process as set forth in claim 84 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

89. The process as set forth in claim 84 wherein the anti-adherent formulation additionally comprises an optional component selected from the group consisting of skin protectants, powders, fragrances, colorants, and preservatives.

90. The process as set forth in claim 84 wherein the base substrate comprises from about 1% (by weight of the base substrate) to about 25% (by weight of the base substrate) of the anti-adherent formulation.

91. The process as set forth in claim 84 wherein the base substrate comprises from about 1% (by weight of the base substrate) to about 10% (by weight of the base substrate) of the anti-adherent formulation.

92. The process as set forth in claim 84 wherein the anti-adherent formulation is encapsulated in a shell material.

93. The process as set forth in claim 92 wherein the shell material comprises a material selected from the group consisting of cellulose-based polymeric materials, carbohydrate-based materials, and materials derived therefrom.

94. The process as set forth in claim 84 wherein the base substrate comprises a material selected from the group consisting of cellulosic fibers and synthetic fibers.

95. The process as set forth in claim 84 wherein the base substrate is a woven material.

96. The process as set forth in claim 84 wherein the base substrate is a non-woven material.

97. The process as set forth in claim 84 wherein the base substrate is a single ply substrate.

98. The process as set forth in claim 84 wherein the base substrate is a multiple ply substrate.

99. The process as set forth in claim 84 wherein both sides of the base substrate comprise the anti-adherent formulation.

100. The process as set forth in claim 84 wherein one side of the base substrate comprises the anti-adherent formulation.

101. The process as set forth in claim 64 wherein the anti-adherent formulation comprises from about 99% (by weight) to about 99.99% (by weight) of fatty acid ester having a melting point greater than 35° C. and from about 0.01% (by weight) to about 1% (by weight) of an anti-adherent compound.

102. The process as set forth in claim 101 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

103. The process as set forth in claim 101 wherein the anti-adherent formulation additionally comprises an optional component selected from the group consisting of skin protectants, powders, fragrances, colorants, and preservatives.

104. The process as set forth in claim 101 wherein the base substrate comprises from about 1% (by weight of the base substrate) to about 25% (by weight of the base substrate) of the anti-adherent formulation.

105. The process as set forth in claim 101 wherein the base substrate comprises from about 1% (by weight of the base substrate) to about 10% (by weight of the base substrate) of the anti-adherent formulation.

106. The process as set forth in claim 101 wherein the anti-adherent formulation is encapsulated in a shell material.

107. The process as set forth in claim 106 wherein the shell material comprises a material selected from the group consisting of cellulose-based polymeric materials, carbohydrate-based materials, and materials derived therefrom.

108. The process as set forth in claim 101 wherein the base substrate comprises a material selected from the group consisting of cellulosic fibers and synthetic fibers.

109. The process as set forth in claim 101 wherein the base substrate is a woven material.

110. The process as set forth in claim 101 wherein the base substrate is a non-woven material.

111. The process as set forth in claim 101 wherein the base substrate is a single ply substrate.

112. The process as set forth in claim 101 wherein the base substrate is a multiple ply substrate.

113. The process as set forth in claim 101 wherein both sides of the base substrate comprise the anti-adherent formulation.

114. The process as set forth in claim 101 wherein one side of the base substrate comprises the anti-adherent formulation.

115. The process as set forth in claim 64 wherein the anti-adherent formulation comprises from about 99% (by weight) to about 99.99% (by weight) of Dow Corning 7-3076 polyamide blend and from about 0.01% (by weight) to about 1% (by weight) of an anti-adherent compound.

116. The process as set forth in claim 115 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

117. The process as set forth in claim 115 wherein the anti-adherent formulation additionally comprises an optional component selected from the group consisting of skin protectants, powders, fragrances, colorants, and preservatives.

118. The process as set forth in claim 115 wherein the base substrate comprises from about 1% (by weight of the base substrate) to about 25% (by weight of the base substrate) of the anti-adherent formulation.

119. The process as set forth in claim 115 wherein the base substrate comprises from about 1% (by weight of the base substrate) to about 10% (by weight of the base substrate) of the anti-adherent formulation.

120. The process as set forth in claim 115 wherein the anti-adherent formulation is encapsulated in a shell material.

121. The process as set forth in claim 120 wherein the shell material comprises a material selected from the group consisting of cellulose-based polymeric materials, carbohydrate-based materials, and materials derived therefrom.

122. The process as set forth in claim 115 wherein the base substrate comprises a material selected from the group consisting of cellulosic fibers and synthetic fibers.

123. The process as set forth in claim 115 wherein the base substrate is a woven material.

124. The process as set forth in claim 115 wherein the base substrate is a non-woven material.

125. The process as set forth in claim 115 wherein the base substrate is a single ply substrate.

126. The process as set forth in claim 115 wherein the base substrate is a multiple ply substrate.

127. The process as set forth in claim 115 wherein both sides of the base substrate comprise the anti-adherent formulation.

128. The process as set forth in claim 115 wherein one side of the base substrate comprises the anti-adherent formulation.
